(19) United States
(12) Patent Application Publication
(10) Pub. No.: US 2005/0163832 A1
(43) Pub. Date: Jul. 28, 2005

(54) Intracellular delivery of therapeutic agents

(76) Inventors: Vladimir Torchilin, Charlestown, MA (US); Ram Rammohan, Boston, MA (US); Tatiana Levchenko, Revere, MA (US); Natalia Volodina, Raynham, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/503,776

(22) PCT Filed: Feb. 13, 2003
(86) PCT No.: PCT/US03/04666

Related U.S. Application Data
(60) Provisional application No. 60/356,526, filed on Feb. 13, 2002.

(51) Int. Cl. A61K 39/21; A61K 39/245; A61K 9/127

(52) U.S. Cl. 424/450; 424/208.1; 424/231.1

(57) ABSTRACT

The preparation and use of a transducing polypeptide (TP)— lipid vesicle complex having a small proportion of positively charged (cationic) lipids in the make-up of the lipid vesicle, e.g., liposome, for safe and efficient intracellular delivery of therapeutic agents, such as proteins, DNA, small molecules and/or other drugs, into a cell of a higher organism, in vitro or in vivo is disclosed. The delivery system of the invention results in increased efficacy of intracellular delivery of such agents, bypassing the endocytotic pathway of intracellular delivery while at the same time minimizing the toxicity of the delivery system towards the recipient cells.

### CROSS REFERENCE TO RELATED APPLICATIONS

This application claims the priority of U.S. Provisional Application No. 60/356,526, filed Feb. 13, 2002, entitled INTRACELLULAR DELIVERY OF DRUGS AND DNA, the whole of which is hereby incorporated by reference herein.

### STATEMENT REGARDING FEDERALLY SPONSORED RESEARCH OR DEVELOPMENT

N/A

### BACKGROUND OF THE INVENTION

Numerous disorders and diseases have been shown to be associated with varying degrees of genetic impairment, such as point mutations, gene deletions or duplications. Thus, the management of these diseases very often requires manipulation at the genetic level, including such possibilities as substitution of the malfunctioning gene(s) or introduction of a multigene complex, usually via the use of “vectors,” which carry a fragment of DNA and are able to replicate within the cell with consequent expression of the protein in question. This process of introducing a gene or DNA into a cell, or transfection, has enormous biotechnological application, but even more so in the ever widening field of medical gene therapy.

Several different methods of DNA delivery into cells are currently available; however, only a few of these are actually in clinical trials or use [Roth et al., (1995); Lisziewicz et al., (1995)]. Common methods such as co-precipitation, membrane permeabilization and electroporation have not been applicable for in vivo use [Song et al., (1995)). Viruses have evolved highly efficient mechanisms of entering cells and reproducing within the host cell. Adenoviruses, retroviruses, and their fragments are, thus, attractive vehicles for gene therapy since they are highly stable, exhibit wide tropism, and can infect quiescent as well as dividing cells (Perricaudet et al., (1995)]. Unfortunately, in spite of their efficiency, they have limitations, including size limit for DNA incorporation, evocation of an immunological response and potential transformation in the host cell [Bett et al., (1993); Yang et al., (1995); Miller et al., (1990)].

Non-viral vector systems include the bombardment with DNA-coated particles, the use of polycations as DNA carriers, and receptor-mediated gene delivery involving complexing plasmid DNA to specific targeting proteins [Rech et al., (1996); Basu, S. K., (1990); Leamon et al., (1991)]. Intensive interest has also focused on amphiphiles such as cationic lipids (liposomes/lipoplexes) as vehicles for the transfer of recombinant genes into a variety of tissues. Liposomes are well known drug carriers with a large capacity for delivering drugs encapsulated into vesicles or incorporated into the membrane [Woodle et al.; (1995), Gergoriadis, G., (1995)]. For many years, liposomes have been investigated as a means for gene delivery. However, to associate the gene material, i.e., negatively charged DNA, with liposomes and to make cells capture liposomes by endocytosis, the liposomes have to be composed using a substantial addition of positively charged lipids, in a quantity which is quite toxic. Commercial Lipofectin® (positively charged liposomes) can be rather successfully used for cell transfection, but the concentration of lipids (and, consequently, the quantity of delivered DNA) is critical in this case, again, because of potential toxicity problems [Scheule et al., (1997); Dokka et al., (2000); Xu et al., (1996)]. Moreover, the usual routes of internalization of drug and DNA carriers by endocytosis or pinocytosis result in subsequent and substantial degradation of intracellularly delivered drugs and DNA by endosomal and lysosomal enzymes, which strongly limits the efficacy of transfection as therapy [Frankel et al., (1988)].

A key limiting factor in the intracellular delivery of drugs and DNA is cell membrane traversability, and numerous approaches have been attempted to overcome this problem. For example, several membrane translocating signal peptides have been described. These include the “protein transduction domains” (PTDs) of HIV-1 TAT protein, the VP22 herpes virus protein and Antennapedia protein (ANTP) [Fawell et al., (1994); Vives et al., (1997); Derossi et al., (1994); Phelan et al., (1998)], which have been shown to efficiently traverse biological membranes. This process is receptor- and transporter-independent, is not endocytosis-mediated and seems to target the lipid layer directly. Many of these peptides promote lipid membrane-reorganizing processes, such as fusion and pore formation, involving temporary membrane destabilization and subsequent reorganization [Prochiantz, A., (1999)]. The minimal PTD of the TAT protein comprises residues 47-57. This and similar peptides derived from TAT protein are termed as TAT peptides. Common structural features of TAT and ANTP PTDs include the presence of many positively charged basic amino acids (arginine and lysine), as well as the ability to adopt an alpha helical conformation. The use of these peptides and protein domains with amphipathic sequences for drug and gene delivery across cellular membranes is getting increasing attention [Fawell et al., (1994); Lindgren et al., (2000); Wagner, E., (1999); Plank et al., (1998); Mi et al., (2000)]. Covalent hitching of proteins, small molecule drugs or DNA onto PTDs may circumvent conventional limitations by allowing the transport of these compounds directly into the cytoplasm of a wide variety of cells in vitro and in vivo. For example, TAT peptide chemically attached to various proteins (e.g., horseradish peroxidase and β-galactosidase) was able to deliver these proteins at high levels to various cells and tissues in the heart, lung and spleen of mice [Schwarze et al., (2000)].

PTDs such as the TAT peptides have been used for intracellular delivery of drug carriers, such as micelles and nanoparticles. For example, dextran-coated iron oxide colloidal particles about 40 nm in diameter and containing several attached molecules of TAT peptide per particle were delivered into lymphocytes much more efficiently than free particles [Lewine et al., (2000)]. Although DNA modified directly with TAT peptide demonstrated good intracellular localization and a good degree of transfection, the direct modification of DNA may be accompanied by various side-effects [Schwarze et al., (2000); Eguchi et al., (2001); Allinquant et al., (1995)].

The internalization by cells of a truncated HIV-1 TAT protein basic domain was shown to proceed at 4° C., i.e., not involving the endocytic pathway [Vives et al., (1997)]. An energy-independent mechanism of translocation through biological membranes was also found for the 60-mer homeodomain of ANTP and was not abolished by directed mutagenesis within the polypeptide C-terminal region; a 16-mer polypeptide derivative with translocating activity was also developed. Again, no classical endocytosis was assumed, since the peptide was effectively internalized at 4° C. [Vives et al., (1997); Derossi et al., (1996)]. Amphipathicity-independent cellular uptake of cell-penetrating peptides also has been shown, and this uptake was also temperature-independent [Derossi et al., (1996)]. Although the actual mechanism of this uptake/translocation has not yet been clearly established, it is suspected that some form of a ligand-lipid interaction plays a role and that direct contact between the translocating moiety and cell membrane is required [Prochiantz, A., (1999)].

Since traversal through cellular membranes represents a major barrier for efficient delivery of macromolecules into cells, the TAT peptide may serve to ferry not only various drugs into mammalian cells in vitro and in vivo, but also larger particles such as liposomes. The efficient intracellular delivery of TAT peptide-modified liposomes was recently demonstrated [Torchilin et al., PNAS (2001)]. Yet, even with this progress, the positively charged lipids of the known liposome systems pose severe toxicity problems for full therapeutic application. Thus, much more progress is required before the development of an efficient and non-toxic system for delivery of drugs and DNA directly to the cytoplasm and into peri-nuclear or nuclear region, bypassing the endosomal pathway, would be possible.

### BRIEF SUMMARY OF THE INVENTION

This invention is directed to the preparation and use of a transducing polypeptide (TP)-lipid vesicle complex having a small proportion of positively charged (cationic) lipids in the make-up of the lipid vesicle, e.g., liposome, for safe and efficient intracellular delivery of therapeutic agents, such as proteins, nucleic acids, small molecules and/or other drugs, into a cell of a higher organism, in vitro or in vivo. The positively charged lipid is in an amount sufficient for complexing with a therapeutic agent but is less than 45 mol % of total lipid in the lipid vesicle, e.g., from 0.01 to 45 mol %, preferably from 0.05 to 35 mol %, more preferably from 0.1 to 25 mol %, even more preferably from 0.5 to 10 mol %, and most preferably about 10 mol % positively charged lipid. Examples of transducing polypeptides include a protein transduction domain of the HIV-1 TAT protein, the VP22 herpes virus protein and the Antennapedia protein (ANTP).

To take advantage of the positive charge on the liposome portion of the delivery system of the invention, it is especially convenient to use as the therapeutic agent a drug molecule bearing a negative charge at the pH value of the liposome preparation. Smaller pieces of nucleic acids, such as RNA, anti-sense RNA, small interfering RNA (siRNA) or mitochondrial DNA (mtDNA), may also be included in the aqueous buffer during liposome preparation and will become trapped in the aqueous core. Very large pieces of (negatively charged) DNA usually cannot be incorporated within the liposome vesicle. Such pieces are preferably complexed via charge interaction with the positively charged, pre-formed liposomes.

The delivery system of the invention results in increased efficacy of intracellular delivery of such agents, bypassing the endocytotic pathway of intracellular delivery while at the same time minimizing the toxicity of the delivery system towards the recipient cells. Also contemplated as within the invention are kits for the preparation of delivery systems or therapeutic compositions according to the invention.

### BRIEF DESCRIPTION OF THE DRAWINGS

Other features and advantages of the invention will be apparent from the following description of the preferred embodiments thereof and from the claims, taken in conjunction with the accompanying drawings, in which:

FIGS. 1A-1D are micrographs showing intracellular trafficking of Rh-PE-labeled and FITC-dextran-loaded TATp-liposomes within BT20 cells. Typical patterns of intracellular localization and integrity of TATp-liposome are shown, after 1 hour (FIG. 1A); 2 hours (FIG. 1B); 4 hours (FIG. 1C); and 9 hours (FIG. 1D). On each figure panel, (a)—DIC (differential interference contrast) light; (b)—DIC with a Rh filter; (c)—DIC with a FITC filter; (d)—DIC composite of (a), (b) and (c), all at x400;

FIG. 2A shows gel-electrophoresis results of free pEGFP-N1 plasmid (1), TATp-liposome/pEGFP-N1 complex (2), and Triton X-100-treated TATp-liposome/pEGFP complex (3); and FIG. 2B shows freeze-etching electron microscopy of TATp-liposomes (a) and TATp-liposome/pEGFP-N1 complex (b);

FIGS. 3A-3B show cell transfection in vitro with TATp-liposome/pEGFP-N1 complexes and TATp-free liposome/pEGFP-N1 complexes. FIG. 3A is a graph displaying flow cytometry data (the number of fluorescent cells and fluorescence intensity on the FITC channel, FL-1H, after 72 hours) for NIH/3T3 cells: (1)—fluorescence of cells treated with TATp-free liposome/pEGFP-N1 complex; (2)—fluorescence of cells treated with an equal quantity (DNA and lipids) of Lipofectin®/pEGFP complex; (3)—fluorescence of cells treated with an equal quantity (DNA and lipids) of TATp-liposome/pEGFP complex. Dotted line shows the position of the peak auto-fluorescense of non-treated cells (negative control); and FIG. 3B shows micrographs (x400, after 72 hours) of NIH/3T3 (a, b) and H9C2 (c, d) cells treated with TATp-liposome/pEGFP-N1 complex. (a) and (c)—bright field light microscopy; (b) and (d)—epifluorescence microscopy with a FITC filter;

FIGS. 4A-4B are bar graphs showing cytotoxicity test results. FIG. 4A shows the comparative cytotoxicity of low-cationic TATp-liposomes according to the invention and Lipofectin® towards NIH/3T3 cells at different lipid concentrations. Incubation was for 24 hrs; cell viability in the presence of 21 μg/ml of TATp-liposomes was taken as 100%. FIG. 4B shows the relative viability of NIH/3T3 cells treated with equal quantities (as DNA, at 5 μg) of TATp-liposome/pEGFP-N1 complex and Lipofectin®/pEGFP-N1 lipoplex. Incubation was for 4 hrs; cell viability in the presence of TATp-liposome/plasmid complex was taken as 100%; and

FIGS. 5a-5f are micrographs (x400) of tissue frozen sections from in vivo growing LLC tumors in mice showing in vivo transfection with TATp-liposome/pEGFP-N1 complex according to the invention. FIG. 5a and FIG. 5b—section from a non-treated tumor (background pattern); FIG. 5c and FIG. 5d—section from the tumor injected with TATp-free liposome/pEGFP-N1 complex; FIG. 5e and FIG. 5f—section from the tumor injected with TATP-liposome/pEGFP-N1 complex. (a), (c), and (e)—bright field light microscopy after hematoxylin/eosin staining; (b), (d), and (f)—epifluorescence microscopy with FITC filter.

### DETAILED DESCRIPTION OF THE INVENTION

This invention is directed to the preparation and use of a transducing polypeptide (TP)-liposome complex having a small proportion of positively charged (cationic) lipids in the make-up of the liposome for safe and efficient intracellular delivery of therapeutic agents, such as proteins, DNA, small molecules and/or other drugs, into a cell of a higher organism, in vitro or in vivo. The delivery system of the invention results in increased efficacy of intracellular delivery of such agents, bypassing the endocytotic pathway of intracellular delivery while at the same time minimizing the toxicity of the delivery system towards the recipient cells.

Preparation of liposomes: Lipid vesicles, especially liposomes, may be prepared by any of the commonly used methods known to those of skill in the art. These include, among others, lipid film hydration, reverse phase evaporation and detergent dialysis (see, e.g., Liposome Technology, G. Gregoriadis, ed., CRC Press, Boca Raton, vol. 1-3, 1984; Phospholipid Handbook, G. Cevc, ed., Marcel Dekker, Inc., New York, 1993; D. D. Lasic, Liposomes. From Physics to Applications, Elsevier, Amsterdam, The Netherlands, 1993).

For example, a lipid film can be formed via solvent evaporation, followed by hydration, using, e.g., cholesterol and phospholipids, such as the neutral lipid phosphatidylcholine, and from 0.01 to 45 mol %, preferably from 0.05 to 35 mol %, more preferably from 0.1 to 25 mol %, even more preferably from 0.5 to 10 mol %, and most preferably about 10 mol % of a positively charged lipid. The lipid combination is dissolved with mixing in chloroform, the solvent is removed by vacuum rotary evaporation, and the resulting lipid film is hydrated in aqueous buffer. This results in the formation of bilayered, membranous, lipid vesicles of various sizes, shapes and aggregate states. If the liposomes need to be sized to obtain a homogeneous distribution, they are extruded, prior to the addition of DNA, through 200 nm size polycarbonate filters. Particle size is estimated by a dynamic light scattering technique.

Attachment of transducing polypeptides: Suitable transducing polypeptides can be directly attached by their reactive groups (such as amino groups, carboxyl groups or sulfhydryl groups) via hydrophobic linkers (which may be polymers such as polyethylene glycol or polyvinylpyrollidine) to preformed lipid vesicle (liposome) membranes. Another alternative is to link the peptides to the liposome surface via the use of a variety of commercially available homo- or hetero-bifunctional reagents known to those of skill in the art (such as carbodiimide, N-succinimidyl(2′-pyridyldithio)propionate (SPDP) or succinimidyl maleidomethyl cyclohexane carboxylate (SMCC), etc.).

For example, as described herein, a TAT peptide (TATp) from the protein transduction domain of the HIV-1 TAT protein was attached to the liposome bilayer by coupling the amino groups of the peptide to p-nitrophenylcarbonyl groups of a linker molecule, such as the polymer (pNP-PEG-PE). This attachment was achieved in two ways. In one method, the linker polymer was included during the formation of the lipid film, the pre-formed liposomes were then incubated with the TATp to allow for coupling, and unbound TATps were removed by gel filtration. In another method, the TATp was first coupled to the linker polymer, unbound TATps were removed by dialysis, and the TAT-pNP-PEG-PE was then included in the lipid film mixture.

Incorporation of therapeutic agents: Therapeutic molecules may be incorporated into the liposomes at different stages of liposome preparation, depending on the physico-chemical properties of the molecules:

(a) Small molecules that can serve as drugs are usually included in the aqueous buffer during liposome formation. Water soluble compounds are trapped in the aqueous core of the vesicles, while hydrophobic molecules distribute mainly into the lipid bilayer. To take advantage of the positive charge on the liposome portion of the delivery system of the invention, it is especially convenient to use a drug molecule bearing a negative charge at the pH value of the liposome preparation, such as sulfathiazole, sulfaoxazol, benzylpenicillin, phenobarbital, sulfacetamide, heparin or acidic proteins and peptides (in order to benefit from the electrostatic attraction to the liposomes). Smaller pieces of nucleic acids, such as RNA, anti-sense RNA, small interfering RNA (siRNA) or mitochondrial DNA (mtDNA), may also be included in the aqueous buffer and will become trapped in the aqueous core.

(b) Very large pieces of (negatively charged) DNA usually cannot be incorporated within the liposome vesicle during lipid film hydration. Such pieces are preferably complexed via charge interaction with positively charged, pre-formed liposomes. For this purpose, positively charged (cationic) lipids, such as DOTMA (2,3-dioleoyloxypropyl-trimetylammonium chloride), a key component of Lipofectin®, and DOTAP (1,2-dioleoyl-3-trimethylammonium-propane), and other positively charged lipids are included in the lipid mixture during liposome preparation, resulting in an array of positive charges on the surface of the fully formed liposome.

Most commercial cationic liposome formulations (such as Lipofectamine® or Lipofectin®) require a large amount (50 mol % or more) of cationic lipid in the composition for optimal DNA complexation and transport. However, the transducing polypeptide-liposome delivery system according to the invention may be prepared with much less positive charge on the surface of the liposome that these prior art systems, and therapeutic agents in therapeutic compositions according to the invention may be delivered into cells of a treated patient with a signficant reduction in toxicity.

The positively charged lipids in the transducing polypeptide-liposome entity complex with the negatively charged DNA, facilitate DNA association, but are not required for the internalization process. The transfer of the whole complex through the cell membrane is mediated by the transducing polypeptides, possibly through the temporary formation of “reversed” micelles in the cell membrane. DNA that can form the above complexes is usually in the form of a plasmid (or, less frequently, an expression vector) that self-replicates in the transfected cell and expresses a protein. Alternatively, the DNA to be transported may be chemically attached to the liposome. DNA in certain forms, such as super-coiled or in small plasmids, may be included inside the liposomes.

In the experiments described herein, the amount of positively charged lipid was about 10 mol %, with the only goal being to enhance complex formation between the negatively charged DNA and the transducing polypeptide (e.g., TATp)-liposomes. The quantity of a positively charged lipid can vary widely, e.g., from 0.01 to 45 mol %, preferably from 0.05 to 35 mol %, more preferably from 0.1 to 25 mol %, and most preferably from 0.5 to 10 mol % of total lipid mixture. In a particular case, the exact sub-toxic quantity of such lipid (i.e., the useful upper end of the mol % range) can be chosen following the determination of the mol % of positively charged lipid necessary for efficient complex formation between the transducing polypeptide-liposome and DNA (i.e., the useful lower end of the mol % range).

The delivery system according to the invention may be loaded with any appropriate therapeutic agent, and the resulting therapeutic compositions according to the invention may be administered to a patient orally, topically, or parenterally (e.g., intranasally, subcutaneously, intramuscularly, intravenously, or intra-arterially) by routine methods in pharmaceutically acceptable inert carrier substances. For example, the compositions of the invention may be administered in a sustained release formulation using a biodegradable, biocompatible polymer, or by on-site delivery using polymeric gels. The therapeutic compositions will be administered in a dosage appropriate for the therapeutic agent being administered. Drug dosages vary widely, e.g., from nanograms per kilogram per day to milligrams per kilogram per day. The delivery system according to the invention is capable of delivering any appropriate dosage desired. Optimal dosage and modes of administration can readily be determined by conventional protocols.

Also contemplated are kits for the preparation of delivery systems or therapeutic compositions according to the invention. Such a kit would include, e.g., transducing polypeptide, pre-modified with linker molecule for incorporation into the liposome bilayer during liposome formation; dry lipid film containing an optimal proportion of positively charged lipid; and physiological buffer. The components are mixed by shaking or vortexing for several minutes to form the transducing polypeptide-liposome delivery system according to the invention. The delivery system can then be complexed with a plasmid containing the appropriate DNA for the therapeutic treatment contemplated. Alternatively, hydrophobic small molecule therapeutic agents may be included in the dry lipid film component of the kit, or hydrophylic therapeutic agents may be included in the buffer component. Then, after the components of the kit are mixed, the resulting therapeutic composition is ready for administration.

The following examples are presented to illustrate the advantages of the present invention and to assist one of ordinary skill in making and using the same. These examples are not intended in any way otherwise to limit the scope of the disclosure.

Materials and Methods

Materials

Egg phosphatidylcholine (PC), cholesterol (Ch), phosphatidyl ethanolamine (PE), polyethylene glycol-PE (PEG-PE), dioleoyl trimethylammonium-propane (DOTAP), and rhodamine-PE (Rh-PE) were purchased from Avanti Polar Lipids. Para-nitrophenylcarbonyl(pNP)-PEG-PE was synthesized in-house. FITC-dextran (MW 4400Da), CL-4B Sepharose, and components of buffer solutions were purchased from Sigma. Lipofectin® Reagent was from Invitrogen. TAT-peptide (11-mer: TyrGlyArgLysLysArgArgGlnArgArgArg; MW 1560 Da) was prepared by ResGen Invitrogen Corporation. Cell culture media—RPMI-1640 (RPMI), Eagle's MEM (EMEM), modified Eagle's medium (DMEM), serum-free medium (Complete Serum-Free), fetal bovine serum (FBS), and heat inactivated FBS were supplied by Cellgro. Fluorescence-free glycerol-based mounting medium (Fluoromount-G) was from Southern Biotechnologies Associates. A pEGFP-N1 plasmid designed for eukaryotic cell expression of the Green Fluorescent Protein (GFP) was obtained from Elim Biopharmaceuticals.

Cell Cultures

Human breast adenocarcinoma cells (BT20) were maintained in EMEM (with 10 mM pyruvate, non-essential amino acids, L-glutamine, and 10% FBS). Lewis lung carcinoma cells (LLC, established from the lung of a C57BL mouse bearing a tumor resulting from an implantation of primary LLC and widely used as a model for metastasis and for studying the mechanisms of cancer chemotherapeutic agents) were maintained in RPMI medium (with 10% FBS). Mouse fibroblasts (NIH/3T3, a continuous cell line of highly contact-inhibited cells, which was established from NIH Swiss mouse embryo cultures in the same manner as the original random bred 3T3 and the inbred BALB/c 3T3; the established NIH/3T3 line was subjected to more than 5 serial cycles of subcloning in order to develop a subclone with morphologic characteristics best suited for transformation assays) and rat cardiomyocytes (H9C2 myoblasts, a subclone of the original clonal cell line derived from embryonic BD1X rat heart tissue exhibiting many of properties of skeletal muscle) were maintained in DMEM (with 10% FBS). Cell lines were from the American Type Culture Collection.

Synthesis of pNP-PEG-PE

pNP-PEG-PE was synthesized according to a published procedure [Torchilin et al., BBA (2001)]. Briefly, 0.1 mmol of PE was dispersed in 8 ml of chloroform supplemented with 2 ml of triethylamine. The resulting mixture was supplemented with 0.5 mmol of PEG₃₅₀₀-(pNP)₂ in 20 ml of chloroform and incubated overnight at room temperature under argon. Organic solvents were removed under vacuum. Dried lipid was dispersed in 0.01 M HCl and purified by gel filtration on CL-4B Sepharose using 0.01 M HCl as an eluent. Pooled fractions containing pNP-PEG-PE were freeze-dried, dissolved in chloroform and stored at −80° C.

Preparation of TATp-Liposomes

A lipid film was prepared by rotary evaporation from a mixture of PC, Ch, and pNP-PEG-PE (7:3:0.05 molar ratio) with traces of Rh-PE in chloroform. This film was re-hydrated in a citrate buffer pH 5.0, vortexed for 5 min, and then extruded through a polycarbonate filter (pore size 200 nm) using an Avanti Mini-Extruder. When loading with FITC-dextran was required, the latter was added as a component of the re-hydration solution. The attachment of TATp to pNP-groups on the liposome surface was carried out by incubating TATp with liposomes in a borate buffer, pH 8.5, overnight at room temperature. The slightly alkaline conditions allow for the coupling reaction with slow hydrolysis of unreacted pNP residues [Torchilin et al., BBA (2001)]. Free FITC-dextran and TATp were removed using a Bio-Gel A15M column. Alternatively, pre-synthesized TAT-PEG-PE conjugate was added to the starting lipid mixture for liposome preparation. The size of FITC-dextran-loaded Rh-labeled TATp-liposomes was measured by dynamic light scattering (DLS) on a Coulter N4 Plus Submicron Particle Analyzer (Coulter Electronics).

Gel-Electrophoresis

Electrophoresis was performed using the E-Gel electrophoresis system from Invitrogen Life Technologies. A pre-cast 0.8% E-Gel cartridge was pre-run for 2 min at 60 V, 500 mA followed by loading of 1 μg of DNA samples in loading dye. Gel running time was approximately 50 min at 60 V, 500 mA. The gel was then photographed over an UV box (Photodyne Technologies).

Freeze-Fracture Electron Microscopy

The sample was quenched using the sandwich technique and liquid nitrogen-cooled propane. A cooling rate of 10,000 Kelvin per second avoids ice crystal formation and artifacts possibly caused by the cryofixation process. The fracturing process was carried out in JEOL JED-9000 freeze-etching equipment (Jeol Inc.) and the exposed fracture planes were shadowed with Pt for 30 sec at an angle of 25-35 degrees and with carbon for 35 sec (2 kV, 60-70 mA, 1×10⁻⁵ Torr). The replicas were cleaned with fuming HNO₃ for 24-to-36 hours followed by repeated agitation with fresh chloroform/methanol (1:1 by vol) at least 5 times, and examined at a JEOL 100 CX electron microscope.

### EXAMPLE I

### Intracellular Trafficking of TATp-Liposomes

Intracellular trafficking and localization of TATp-liposomes were tested in BT20 cells grown on coverslips in 6-well plates. At approximately 60-70% confluency, cells were incubated with liposomes in a serum-free medium at 37° C. under 6% CO₂. The medium was removed and the cells washed with sterile PBS, pH 7.4, after 1, 2, 4, 9 and 24 hr incubation. Coverslips were mounted cell-side down with fluorescence-free glycerol-based mounting medium and viewed by epi-fluorescence microscopy (Nicon Eclipse E400, Nicon Co.) and deconvolution differential interference contrast (DIC) microscopy with pseudo-coloring (Axioplan 2, Zeiss Co.).

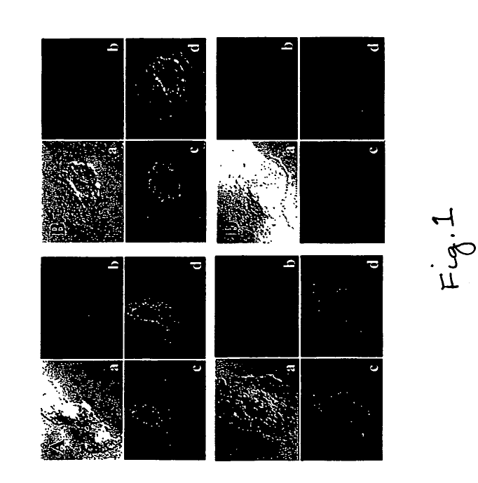

Free FITC-dextran showed only minimal intracellular accumulation in the BT20 cells used (not shown), while 200 nm Rh-labeled TATp-liposomes loaded with FITC-dextran rapidly translocated into these cells. Typical patterns of time-dependent distribution of TATp-liposomes inside individual cells are shown in FIGS. 1A-1D. After 1 hour, their diffuse localization within the cell cytoplasm was evident (FIG. 1A). Intracellular liposomes apparently remained intact within this time period, since the flourescence of the intraliposomal (FITC-dextran) and membrane (Rh-PE) labels coincided. With time, TATp-liposomes, similar to TATp (37,44), gradually migrated closer to the nuclei, and after 2 and 4 hours, a significant fraction of TATp-liposomes was seen surrounding the peri-nuclear region, with a reduced cytoplasmic distribution (FIGS. 1B and 1C). At 9 hours, the degradation of liposomes was observed (diffuse orange/red fluorescence in the cytoplasm and nucleus) with some liposomes remaining in the peri-nuclear region. However, by this time the FITC-dextran was almost totally released (diffuse green fluorescence) (FIG. 1D). By hour 24, virtually no internal or membrane label could be seen inside the cells.

These experiments clearly showed that, in good agreement with earlier observations [Torchilin et al., PNAS (2001)], the uptake of TATp-liposomes is fast and efficient. Because of the nuclear tropism imparted by the TATp [Vives et al., (1997); Truant et al., (1999)] TATp conjugation allows for a gradual peri-nuclear localization of liposomes, bypassing the endocytic pathway. Eventually, liposomes are destroyed and release their contents. The relatively slow peri-nuclear accumulation of TATp-liposomes compared to free TATp may be explained by hindered diffusion of larger liposomal particles in the cytoplasm.

### EXAMPLE II

### Preparation of TATp-Liposome/Plasmid Complexes

Liposomes for complexation with DNA did not contain any fluorescent labels, but did contain up to 10 mol % of the cationic lipid DOTAP to enhance plasmid association. Liposomes from a mixture of PC, Ch, DOTAP, and pNP-PEG-PE (7:3:1:0.05 molar ratio) were prepared as above, and incubated with the pEGFP-N1 plasmid overnight at 4° C. In a typical case, the liposome/plasmid complex containing a total of 2 mg lipid and 200 μg DNA was incubated with an appropriate amount of TATp overnight at pH 8.5 in a borate buffer, and purified by gel filtration on Bio-Gel A-1.5. The post-column fraction was subjected to agarose gel electrophoresis to test for the presence and intactness of the plasmid in complex with the liposomes. To determine DNA content, the post-column TATp-liposome/plasmid complex-containing fraction was treated with Triton X-100 for 1 hour at 37° C. to release the plasmid from the complex, and then subjected to agarose gel electrophoresis. Lipofectin®/pEGFP-N1 complex was prepared according to the manufacturer's instruction (Invitrogen Corp.) using same quantities and ratios of lipid and DNA (which are within the recommended limits for this preparation).

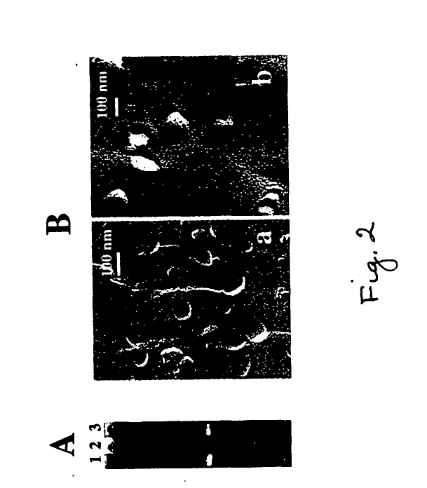

TATp-liposomes with a relatively low (≦10 mol %) content of a cationic DOTAP effectively complexed and firmly retained intact plasmid as evidenced by the gel electrophoresis data (FIG. 2A). TATp-liposome/pEGFP-N1 complexes could not enter a gel because of their large size (FIG. 2A, line 2). However, after Triton-X100 treatment, all complexed DNA was released in a free form, resulting in a band with intensity close to the control free DNA (compare lines 1 and 3 on FIG. 2A). Depending on the particular need, the total quantity of DNA in a sample could vary, e.g., from 0.05 to 0.2 μg per 1 μg of lipid, which is similar to what is normally achieved with Lipofectin® (according to manufacturer's instructions). Complexation of a plasmid with liposomes only moderately increased their size (from ca. 150 to ca. 200 nm by DLS). The freeze-etching electron microscopy also showed that the major fraction of TATp-liposome/plasmid complexes maintained an essentially spherical shape with a size of about 200 nm (FIG. 2B). Both preparations displayed convex and concave fracture planes typical of liposomal structure. The complexes were non-aggregated. Their boundaries appeared sometimes dotted—a phenomenon also observed in other liposome/nucleic acid complexes [J\check{a}\check{a}skel\check{a}inen et al., (1998)]. Thus, TATp-liposomes with a low content of a cationic lipid can complex and retain substantial quantities of DNA.

### EXAMPLE III

### Transfection In Vitro

NIH/3T3 or H9C2 cells grown to 60-70% confluency on coverslips were incubated in serum-free media with TATp-liposome/plasmid complexes or liposome/plasmid complexes (in the quantity required to deliver 5 μg DNA per 200,000 cells at DNA concentration of 0.3 μg/μl of added liposomal suspension) for 4 hrs at 37° C. under 6% CO₂. The same quantity of Lipofectin®/pEGFP-N1 complex with the same lipid-to-DNA ratio was used as the control. After incubation, the medium was removed and the cells were washed twice with sterile PBS and re-incubated with complete DMEM containing 10% FBS for 72 hrs. For flow cytometry (FACScan™, Becton Dickinson Biosciences), NIH/3T3 cells were grown in 25 cm² flasks and fixed in 4% paraformaldehyde. GFP expression was visualized by light microscopy and epifluorescence microscopy using a FITC filter.

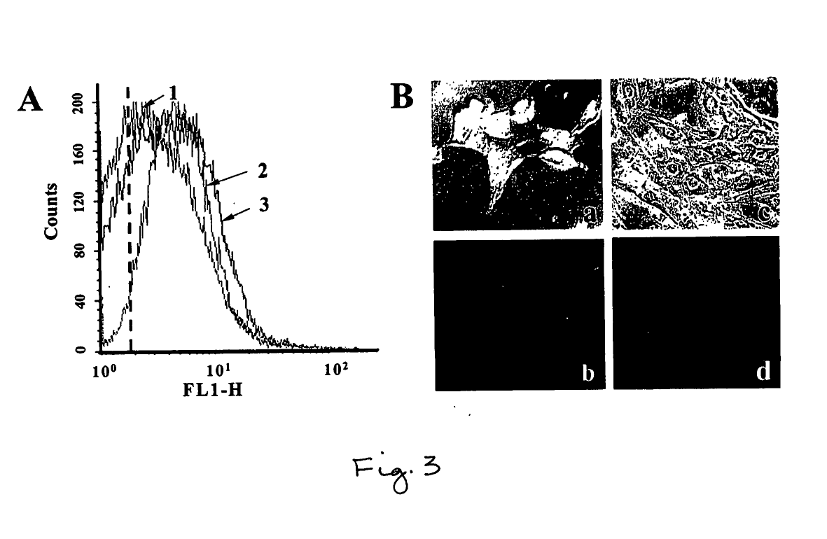

The results of the treatment of NIH/3T3 fibroblasts and H9C2 cardiomyocytes with TATp-liposome/pEGFP-N1 complexes are presented in FIGS. 3A-3B. The flow cytometry data (FIG. 3A) show that the treatment of NIH/3T3 cells with TATp-free liposome/pEGFP-N1 complexes results in a slight increase in cell fluorescence (compare the position of the curve 1 peak on FIG. 3A with the dotted line showing the peak autofluorescence on non-treated cells). This fluorescense may result from some cell transfection via non-specifically captured plasmid-bearing liposomes. At the same time, cells treated with TATp-liposome/pEGFP-N1 complexes demonstrated a substantially higher fluorescence (curve 3 vs. curve 1 on FIG. 3A), i.e., a higher transfection outcome. At similar conditions, Lipofectin®/pEGFP-N1 complexes provided essentially the same extent of transfection and fluorescence level as TATp-liposome/plasmid complexes (curve 2 on FIG. 3A).

Confocal microscopy confirmed the transfection of both NIH/3T3 and H9C2 cells with TATp-liposome/DNA complexes (FIG. 3B). From 30 to 50% of both cell types in the field of view show a bright green fluorescence, while lower fluorescence was observed in virtually all cells. As can be seen from the photographs, the transfection was not accompanied by any visible toxic effects. All cells appear morphologically normal.

### EXAMPLE IV

### Cytotoxicity Assay

NIH/3T3 cells were seeded in 96-well tissue culture microtiter plates. After 24 hrs, the culture medium was removed and the cells were treated with TATp-liposomes, Lipofectin®, TATp-liposome/pEGFP-N1 complex, or Lipofectin®/pEGFP-N1 complex in serum-free medium. The experiments were carried out both in the absence of the plasmid at several different concentrations of low-cationic TATp-liposomes and Lipofectin®, and in the presence of plasmid, at concentrations of TATp-liposomes and Lipofectin® required to provide DNA concentration of 5 μg/ml (total lipid concentration for both preparations varied from approximately 20 to approximately 100 μg/ml). After 24 hrs in case of plasmid-free preparations and after 4 hrs in case of DNA-containing preparations, the medium was removed, CellTiter 96 Aqueous One solution (Promega) added to each well, and the plates re-incubated for 4 hrs. This assay is based on the bioreduction of MTS tetrazolium compound (Owen's reagent) into a colored soluble formazan product. The viability of cells was measured using a plate-reader (Multiscan MCC/340, Fisher Scientific) at 490 nm. Relative viability was calculated with cells treated only with medium alone as a control. The statistical treatment of the data was performed according to the Student's T test for two populations.

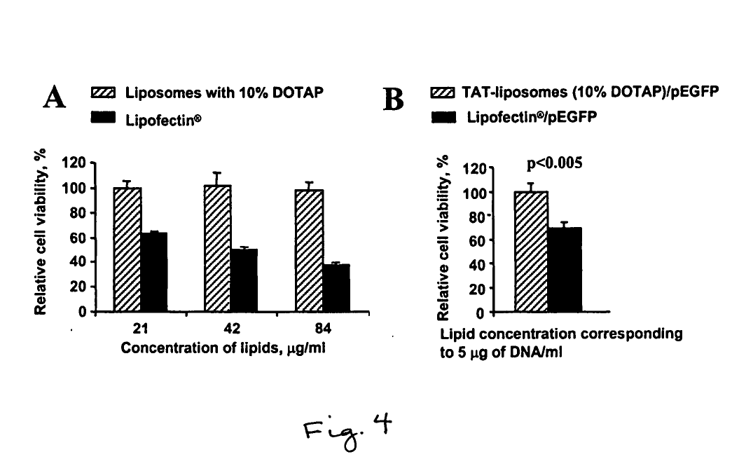

As can be seen in FIG. 4A, at similar concentrations, low-cationic TATp-liposomes with 10 mol % of DOTAP were non-toxic for the cells even after 24 hrs of incubation, while the same quantities of Lipofectin caused the death of 35 to 65% of cells in a concentration-dependent fashion. This observation was true for any lipid concentration over the range between 20 and 80 μg/ml. In the experiment performed with TATp-liposome/plasmid complex using the same quantity of Lipofectin®-plasmid lipoplex as a control (5 μg DNA and 20 μg of lipid per ml in both cases, which is a normal working concentration for the Lipofection procedure), the TATp-liposome/plasmid complex was about 25% less toxic than the Lipofectin®-plasmid lipoplex to the NIH/3T3 cells after only 4 hrs of incubation (FIG. 4B). Thus, complexes according to the invention of DNA with TATp-liposomes with a low content of a positively charged lipid can complex and deliver DNA into cells with less toxic effects than is typical for many non-viral DNA delivery systems with a high content of positive charge.

### EXAMPLE V

### Transfection In Vivo

The aim of this study was to carry out an in vivo transfection in a whole animal model with Lewis lung carcinoma cells (LLC). Transfection was localized by direct administration of TATp-liposome/pEGFP-N1 complexes into the tumor tissue to minimize the non-specific transfection of other tissues.

LLC tumors were grown in C57BL/6 mice (Charles River Laboratories) by subcutaneous injection of 8×10⁴ LLC cells per mouse into the left flank (protocol 011022 approved by the IACUC, Northeastern University, Nov. 26, 2001). Tumors were injected at 4-5 different spots with 100 μl of TATp-liposome/pEGFP-N1 complex in HBS, pH 7.4, after they reached 5-to-10 mm in diameter. Mice were sacrificed at 72 hr later by cervical dislocation, and excised tumors were fixed in a 4% buffered paraformaldehyde overnight at 4° C., blotted dry of excess paraformaldehyde and kept in 20% sucrose in PBS overnight at 4° C. Cryofixation was done by immersion of tissues in ice cold isopentane for 3 min followed by freezing at −80° C. Fixed, frozen tumors were mounted in Tissue-Tek OCT 4583 compound (Sakura Finetek USA) and sectioned on a Leica Jung Frigocut 2800N (Leica Instrument). Sections were mounted on slides and analyzed by fluorescence microscopy and with hematoxylin/eosin staining. Tumor-bearing mice injected with TATp-free liposome/plasmid complexes of the same composition and non-injected mice with similar-sized tumors were used as negative controls.

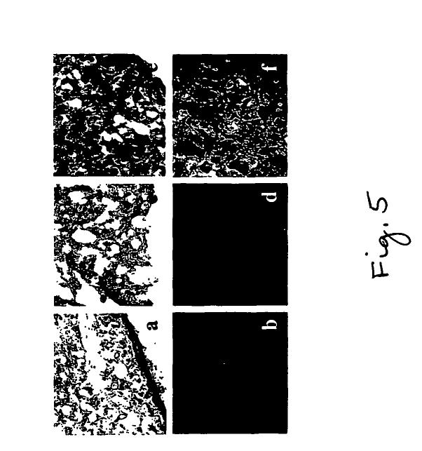

FIG. 5 presents the in vivo results with LLC-bearing mice. Histologically, hematoxylin/eosin-stained tumor slices in both control and experimental animals showed a typical pattern of poorly differentiated carcinoma (polymorphic cells with basophilic nuclei forming nests and sheets and containing multiple sites of neoangiogenesis; FIGS. 5a, 5c, and 5e). Under the fluorescence microscope, samples from control mice (non-treated mice or mice injected with TATp-free liposome/plasmid complexes; FIGS. 5b and 5d) showed only a background fluorescence, while slices from tumors injected with TATp-liposome/plasmid complexes contained bright green fluorescence in tumor cells (FIG. 5f) indicating the TATp-mediated transfection in vivo.

### REFERENCES

Allinquant, B., Hantraye, P., Mailleux, P., et al. Downregulation of amyloid precursor protein inhibits neurite outgrowth in vitro. J. Cell. Biol. 128, 919-27 (1995).
Basu, S. K. Biochem. Pharmacol. 40, 1941-1946 (1990).
Bett, A. J., Prevec, L., and Graham, F. L. Packaging capacity and stability of human adenovirus type 5 vectors. J. Virol. 67, 5911-5921 (1993).
Derossi, D., Joliot, A. H., Chassaing, G., and Prochaintz, A. Cell internalization of the third helix of the Antennapedia homeodomain is receptor-independent. J. Biol. Chem. 271, 18188-18193 (1996).
Derossi, D., Joliot, A. H., Chassaing, G., and Prochaintz, A. The third helix of the Antennapedia homeodomain translocates through biological membranes. J. Biol. Chem. 269, 10444-10450 (1994).
Dokka, S., Toledo, D., Shi, X., Castranova, V., and Rojanasakul, Y. Oxygen radical-mediated pulmonary toxicity induced by some cationic liposomes. Pharm. Res. 17, 521-525 (2000).
Eguchi, A., Akuta, T., Okuyama, H., Senda, T., Yokou, H., Inokuchi, H., Fugjita, S., Hayakawe, T., Hasegawa, M., Nakanishi, M. Protein transduction domain of HIV-1 Tat protein promotes efficient delivery of DNA into mammalian cells. J. Biol. Cem. 276, 26204-26210 (2001).
Fawell, S., Seery, J., Daikh, Y., Moore, C., et al. TAT-mediated delivery of heterologous proteins into cells. Proc. Nat'l Acad. Sci. U.S.A. 91, 664-668 (1994).
Frankel, A. D., and Pabo, C. O. Cellular uptake of the TAT protein from human immunodeficiency virus. Cell 55, 1189-1193 (1988).
Gregoriadis, G. Engineering liposomes for drug delivery: progress and problems. Tibitech 13, 527-537 (1995).
Jääskeläinen, I., Stenberg, B. J. Mökkönen, J., & Urtti, A. Int. J. Pharm. 167, 1991-203 (1998).
Leamon, C. P., and Low, P. S. Proc. Nat'l Acad. Sci. U.S.A. 88, 5572-5576 (1991).
Lewin, M., Carlesso, N., Tung, C.-H., Tang, X.-W., Cory, D., Scadden, D. T., Weissleder, R. Tat peptide-derivatized magnetic nanoparticles allow in vivo tracking and recovery of progenitor cells. Nature Biotech. 18, 410-414 (2000).
Lindgren, M., Hallbrink, M., Prochiantz, A., and Langel, U. Cell-penetrating peptides. Trends Pharmacol. Sci. 21, 99-103 (2000).
Lisziewicz, J., Sun, D., Lisziewicz, A., and Gallo, R. C. Antitat gene therapy: a candidate for late-stage AIDS patients. Gene Ther. 2, 218-222 (1995).
Mi, Z., Mai, J., Lu, X. Robbins, P. D. Characterization of a class of cationic peptides able to facilitate efficient protein transduction in vitro and in vivo. Mol. Ther. 2, 339-347 (2000).
Miller, D. G., Adam, M. A., and Miller A. D. Gene transfer by retrovirus vectors occurs only in cells that are actively replicating at the time of infection. Mol. Cell Biol. 10, 4239-4242 (1990).
Perricaudet, M., and Stratford-Perricaudet, L. D. Ch.1 Adenovirus-mediated in vivo gene therapy. In “Viruses in Human Gene Therapy.” (Vos, J. M. H., ed), pp 1-32. Chapman and Hall, London (1995).
Phelan, A., Elliott, G., and O'Hare, P., Intercellular delivery of functional p53 by the herpes virus protein VP22. Nat. Biotechnol. 16, 440-443 (1998).
Plank, C., Zauner, W., Wagner, E. Application of membrane-active peptides for drug and gene delivery across cellular membranes. Adv. Drug Deliv. Rev. 34, 21-35 (1998).
Prochiantz, A. Homeodomain-derived peptides. In and out of the cells. Ann. NY Acad. Sci. 886, 172-179 (1999).
Rech, E. L., De-Bem, A. R., Aragao, F. J. Biolostic-mediated gene expression in guinea pigs and cattle tissues in vivo. Braz. J. Med. Biol. Res. 29, 1265-1267 (1996).
Roth, J. A., Swisher, S. G., Merritt, J. A., et al. Gene therapy for non-small cell lung cancer: a preliminary report of phase I trial of adenoviral p53 gene replacement. Semin. Oncol. 25 (3 Suppl 8), 33-37 (1998).
Scheule, R. K., St. George, J. A., Bagley, R. G., et al. Basis of pulmonary toxicity associated with cationic lipid-mediated gene transfer to the mammalian lung. Hum. Gene Ther. 8, 689-707 (1997).
Schwarze, S. R., Dowdy, S. F. In vivo protein transduction: intracellular delivery of biologically active proteins, compounds and DNA. TIPS 21, 45-48 (2000).
Schwarze, S. R., Ho, A., Vocero-Akbani, A., Dowdy, S. F. In vivo protein transduction: delivery of a biologically active protein into the mouse. Science 285, 1569-1572 (1999).
Song, W., and Lahiri, D. K. Efficient transfection of DNA by mixing cells in suspension with calcium phosphate, Nucleic Acids Res. 23, 3609-3611 (1995).
Torchilin, V. P., Levchenko, T. S., Lukyanov, A. N., Khaw, B. A., Klibanov, A. L., Rammohan, R., Samokhin, G. P., & Whiteman, K. R. Biochim. Biophys. Acta 1511, 397-411 (2001).
Torchilin, V. P., Rammohan, R., Weissig, V., and Levchenko, T. S. TAT peptide on the surface of liposomes affords their efficient intracellular delivery even at low temperature and in the presence of metabolic inhibitors. Proc. Nat'l Acad. Sci. U.S.A. 98, 8786-8791 (2001).
Truant, R. & Cullen, B. R. Mol. Cell. Biol. 19, 1210-1217 (1999).
Vives, E., Brodin, P., and Lebleu, B. A truncated HIV-1 TAT protein basic domain rapidly translocates through the plasma membrane and accumulates in the cell nucleus. J. Biol. Chem. 272, 16010-16017 (1997).
Wagner, E. Application of membrane-active peptides for nonviral gene delivery. Adv. Drug Deliv. Rev. 38, 279-289 (1999).
Woodle, M. C., Newman, M. S., and Working, P. K. Ch 10. Biological properties of sterically stabilized liposomes. In “Stealth Liposomes” (Lasic, L., and Martin, F., eds) CRC Press, Inc. (1995).
Xu, Y., and Szoka, F. C. Jr. Mechanism of DNA release from cationic liposome/DNA complexes used in cell transfection. Biochemistry 35, 5616-5623 (1996).
Yang, Y., Li, Q., Ertl, H. C. J., and Wilson, J. Cellular and humoral immune responses to viral antigens create barriers to lung-directed gene therapy with recombinant adenoviruses. J. Virol. 69, 2004-2015 (1995).

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

1. A delivery system for a therapeutic agent, said system comprising:
a lipid vesicle complexed with one or more transducing polypeptides, said lipid vesicle comprising positively charged lipid,
wherein said positively charged lipid is in an amount sufficient for complexing with a therapeutic agent but less than 45 mol % of total lipid in said lipid vesicle.

2. The delivery system of claim 1, wherein said lipid vesicle comprises between 0.01 and 45 mol % positively charged lipid.

3. The delivery system of claim 1, wherein said lipid vesicle comprises between 0.05 and 35 mol % positively charged lipid.

4. The delivery system of claim 1, wherein said lipid vesicle comprises between 0.1 and 25 mol % positively charged lipid.

5. The delivery system of claim 1, wherein said lipid vesicle comprises between 0.5 and 10 mol % positively charged lipid.

6. The delivery system of claim 1, wherein said lipid vesicle comprises about 10 mol % positively charged lipid.

7. The delivery system of claim 1, wherein said transducing polypeptide is a protein transduction domain of the HIV-1 TAT protein.

8. The delivery system of claim 1, wherein said transducing polypeptide is the VP22 herpes virus protein.

9. The delivery system of claim 1, wherein said transducing polypeptide is the Antennapedia protein (ANTP).

10. The delivery system of claim 1, wherein said positively charged lipid is 2,3-dioleoyloxypropyl-trimetylammonium chloride or 1,2-dioleoyl-3-trimethylammonium-propane.

11. The delivery system of claim 1, wherein said transducing polypeptide is complexed with said lipid vesicle via a linker molecule.

12. The delivery system of claim 11, wherein said linker molecule is a polymer.

13. The delivery system of claim 11, wherein said linker polymer is attached to said lipid vesicle during lipid vesicle formation.

14. A therapeutic composition comprising
the delivery system of claim 1; and
a therapeutic agent complexed with said delivery system.

15. The therapeutic composition of claim 14, wherein said therapeutic agent is nucleic acid.

16. The therapeutic composition of claim 15, wherein said nucleic acid is DNA.

17. The therapeutic composition of claim 16, wherein said DNA is in the form of a plasmid.

18. The therapeutic composition of claim 16, wherein said DNA is in the form of an expression vector.

19. The therapeutic composition of claim 14, wherein said therapeutic agent is RNA.

20. The therapeutic composition of claim 14, wherein said therapeutic agent is anti-sense RNA.

21. The therapeutic composition of claim 14, wherein said therapeutic agent is small interfering RNA.

22. The therapeutic composition of claim 14, wherein said therapeutic agent is mitochondrial DNA.

23. The therapeutic composition of claim 14, wherein said therapeutic agent is a small molecule drug.

24. The therapeutic composition of claim 14, wherein said therapeutic agent is complexed with the external surface of said lipid vesicle.

25. The therapeutic composition of claim 14, said lipid vesicle having an aqueous core, and wherein said therapeutic agent is in said aqueous core.

26. The therapeutic composition of claim 14, said lipid vesicle having a lipid bilayer membrane, and wherein said therapeutic agent is distributed in said lipid bilayer membrane.

27. A method of treating a patient in need of therapy, said method comprising
providing a patient in need of therapy; and
administering to said patient a therapeutically effective amount of the therapeutic composition of claim 14.

28. A kit for preparing a delivery system for a therapeutic agent, said kit comprising
a transducing polypeptide;
a mixture of dry lipids, said mixture comprising positively charged lipid, wherein said positively charged lipid is in an amount sufficient for complexing with a therapeutic agent but less than 45 mol % of total lipid in said lipid vesicle; and
physiological buffer.

29. The kit of claim 28, wherein said transducing polypeptide is modified with a linker molecule.

30. A kit for preparing a therapeutic composition, said kit comprising
the kit of claim 28, wherein said dry lipid mixture comprises a hydrophobic therapeutic agent.

31. A kit for preparing a therapeutic composition, said kit comprising
the kit of claim 28, wherein said physiological buffer comprises a hyrophylic therapeutic agent.
